(19) United States
(12) Patent Application Publication
(10) Pub. No.: US 2021/0205070 A1
(43) Pub. Date: Jul. 8, 2021

(54) INTRAOCULAR IMPLANT AND METHOD FOR PRODUCING AN INTRAOCULAR IMPLANT

(71) Applicant: Carl Zeiss Meditec AG, Jena (DE)

(72) Inventors: Manfred Dick, Gefell (DE); Mark Bischoff, Jena (DE)

(21) Appl. No.: 17/190,514

(22) Filed: Mar. 3, 2021

Related U.S. Application Data
(63) Continuation-in-part of application No. PCT/EP2019/073340, filed on Sep. 2, 2019.

(30) Foreign Application Priority Data
Sep. 7, 2018 (DE) 10 2018 215 258.6
Dec. 17, 2018 (DE) 10 2018 221 947.8

(51) Int. Cl. A61F 2/16; A61F 2/14; A61L 27/38; A61L 27/36; A61L 27/24

(52) U.S. Cl. CPC A61F 2/1601; A61F 2/1451; A61F 2240/002; A61L 27/3641; A61L 27/24; A61L 27/3804

(57) ABSTRACT

An intraocular implant, such as a corneal implant, an intraocular lens or an IOL carrier matrix, which has a dimensionally stable lattice structure and a corresponding method for producing an intraocular implant. The intraocular implant and method for producing an intraocular implant counteract metabolic problems, and thus also limited functional compatibility, and facilitates long-term tolerance. The intraocular implant has a dimensionally stable lattice structure designed in such a way that it permits permeability for small molecules and/or supports the mobility of endogenous cells in the implant.

### CROSS-REFERENCE TO RELATED APPLICATIONS

This application is a continuation-in-part under 35 USC § 111(a) of PCT Application PCT/EP2019/073340, filed Sep. 2, 2019 and entitled “Intraocular Implant and Method for Producing an Intraocular Implant,” which claims the benefit of priority to DE Application No. DE 10 2018 215 258.6, filed Sep. 7, 2018 and DE Application No. 10 2018 221 947.8, filed Dec. 17, 2018, the entire contents of all of which are hereby incorporated by reference herein.

### TECHNICAL FIELD

Embodiments of the present invention relate to an intraocular implant, in particular a corneal implant, an intraocular lens or an IOL carrier matrix, which has a dimensionally stable lattice structure. The present invention also relates to a corresponding method for producing an intraocular implant of this type.

### BACKGROUND

Intraocular implants, in particular corneal implants, are used in different designs for implantation in the cornea of the human eye in order to rectify serious visual impairment of a patient. It has been found to be problematic that all implants in the stroma of the cornea, or quite generally within the cornea, disturb the metabolism in the long term and, consequently, do not offer a permanent solution. Attempts have already been made to solve the problem by using very small, thin and as far as possible permeable biocompatible transparent materials or finely perforated inserts, but these attempts have thus far been only partially successful. In this context, implants are said to have a limited functional compatibility, as distinct from biocompatibility. The problem of limited functional compatibility can also be applied to a greater or lesser extent to other tissue implants in the eye.

By contrast, the functional compatibility of transplants, i.e. of other tissue from the body of the recipient or from another organism, is generally quite good, for which reason corneal transplants have become an important tool in the treatment of serious diseases of the cornea. Certain transplant methods are surgically so simple, and to all intents and purposes clinically effective and safe, that they can also be considered for surgical correction of visual impairments. Particularly in the case of autologous transplants, i.e. where the donor and the recipient are the same person, there is a very high probability of the compatibility of the transplants lasting for an unlimited time. However, such a solution is not possible in every case, which is why there is still a great need for (synthetically produced) implants.

For the cornea again, the biosynthetic production of cornea replacements, i.e. corresponding corneal implants based on hydrogels, is also known. Although good transparency in the visible spectral range can be achieved by this approach, there are in particular mechanical properties that are disadvantageous compared to natural tissue.

US 2016/0325499 A1 describes a system and a method for the production of a tissue-related structure by a three-dimensional printing method. Said document also mentions the printing of corneal implants with a corneal collagen matrix that contains keratocytes added during printing. An approach is shown for generating implants of this kind, here biocompatible implants, in a simple manner. Biocompatibility is guaranteed. However, implants produced in this way may also still disturb the metabolism, which therefore casts doubt on their long-term tolerance.

### SUMMARY

Embodiments of the present invention include an intraocular implant, and a method for producing an intraocular implant, which counteracts corresponding metabolic problems, and thus also limited functional compatibility, and guarantees long-term tolerance.

An intraocular implant for implantation in an eye structure, particularly into the optically active zones of the eye, has a dimensionally stable lattice structure. According to the invention, this dimensionally stable lattice structure is designed in such a way that it permits permeability for small molecules (in any form!) and/or supports the mobility of endogenous cells in the implant, for example into the implant in order to colonize the latter, or through the implant.

Examples of the small molecules in question are water for all structures of the eye, intrastromal or corneal fluid, especially for a corneal implant, or intraocular fluids in general. The permeability of the intraocular implant is substantially ensured by a corresponding lattice structure. The dimensionally stable lattice structure is thus designed such that a metabolism comparable to the natural tissue can take place. Although helpful for this purpose, it is not absolutely necessary to use the material that corresponds to the material of the natural tissue structure. Of more importance is a biocompatible (generally organic) material, hence a material compatible with the natural tissue of the eye, which substantially prevents rejection of the implant by the organism, and at the same time a substantially transparent material which is formed in such a structure so that the natural metabolic processes can be simulated in their entirety. The permeability of the lattice structure for small molecules, in particular for the characteristic molecules present on this structure, is one requirement; the mobility of endogenous cells in the implant is a further requirement. According to the invention, both requirements are taken specifically into account depending on the structure that is to be implanted.

The main focus here is on the corneal implant, since the latter is the most widely used and the one where corresponding improvements are the most striking. Except for a few properties that relate specifically to the cornea, the herein described properties of particular embodiments of the corneal implant are also applicable to other intraocular implants. Examples of other intraocular implants are in particular a corresponding intraocular lens and support structures for same.

The invention therefore relates in a more general sense to an intraocular implant for implantation in an eye structure, i.e. in the cornea, but also in deeper-lying structures of the eye. The distinction between transplant and implant will be underlined once again here. While the material of transplants relates to tissue from the body of the recipient or from another organism, as has already been described, implants within the meaning of the invention are distinguished by the fact that they are composed of an artificially generated material. The latter can also be a material that has been generated from explanted tissue, for example by decellularization or fragmentation.

The permeability of the intraocular implant according to the invention, in particular of the corneal implant according to the invention, for small molecules is achieved by a suitably formed lattice structure and is adapted to the behavior of a corresponding natural structure and, along with the mobility of endogenous cells, prevents degradation of the surrounding tissue over time. A reduced metabolism in fact leads to the accumulation of corresponding molecules (e.g. liquids) that would have been transported away in the natural eye tissue and thus leads in the long term to impairment or even destruction of the tissue or of the implant. According to the invention, by use of suitable structuring, the permeability is therefore significantly increased in relation to conventional intraocular implants, in particular corneal implants.

As has already been mentioned, the intraocular implant according to the invention has a high level of dimensional stability in the deployed state—with a stable but elastic internal lattice structure, i.e. a more or less regular inner structure, that permits the permeability for small molecules and/or supports the mobility of endogenous cells in the implant—but is flexible during insertion. The term lattice structure should not be understood too narrowly here in the sense of solid-state physics, but a microscopically irregular framework structure should also be possible. Such irregularity can in particular be useful to avoid diffraction effects.

This may even comprise lattice structures which are designed such that starting from a (virtual) regular lattice with constant lattice distances, each lattice distance (corresponding to the dimension of a smallest unit of this lattice) is blurred by a random value.

An intraocular implant is advantageous that, for example, comprises channels with a diameter of greater than 1 μm, for example greater than 3 μm, and webs with a dimension of less than 50 μm, for example less than 20 μm, for their diameters. This optimally supports a permeability for corresponding tissue fluids of the eye that are to be transported away, and it even permits the movement of cells through the outer surface of the implant and within the implant.

The channels do not necessarily have to extend all the way through the whole structure of the implant from the inside to the outside. Rather, a plurality of subsections that are offset in relation to one another can be used in this sense. The subsections of the channels can also vary in terms of their direction, i.e. it is not always necessary to provide the fastest or most direct route.

Although a certain minimum dimension of the webs contributes greatly to the stability of the lattice structure of the intraocular implant, the dimension or diameter of the webs should not be so large as to locally prevent the permeability for the corresponding molecules. This results in the values suggested above.

Another example is an intraocular implant that contains a biocompatible material, for example a collagen, which is arranged in the form of fibrils, i.e. in a fibrous or filamentary form, in lamellae in the lattice structure.

Collagens are a natural, substantially organic constituent of the cornea, but also of other eye tissue. They can be obtained by technical approaches. They are also suitable for an additive production method. Therefore, they naturally represent a suitable base material for intraocular implants.

Also of advantage is an intraocular implant whose lamellae are alternately arranged at right angles to respective neighboring lamellae and in which the lamellae are also interconnected, for example interwoven. An intraocular implant of this kind is ideally of a layered construction and contains microfibril bundles of differing density which are for example fixed at the periphery.

Natural structures are “simulated” in this way. The cornea consists naturally of dense, regularly connected tissue. The collagen of the natural eye structure is arranged in lamellae with the fibrils, wherein the lamellae are alternately oriented at right angles to the neighboring lamellae. A simulation of this structure is provided, according to the invention, in particular by additive manufacture.

It is a property of human corneal tissue that bundles of microfibrils of different density are incorporated therein. These are important for the stability and elasticity of the material and are therefore for example replicated in the implant. According to the invention, fiber-like elements then run in layers (laterally) diagonally through the implant. They are fixed in the periphery. There, they for example have an increased elasticity, which should be less in the central region of the implant. The bundles of microfibrils form a braid that has substantially a lamellar structure.

The shape of a lattice of microfibers and filaments of collagen material is one possible example of the lattice structure of the intraocular implant according to the invention.

Said collagen structure is naturally colonized only sparingly with keratocytes. Therefore, the implant is for example not artificially colonized with cells. However, endogenous cells of this kind are able to move into an intraocular implant according to the invention.

In a particular embodiment of the intraocular implant according to the invention, said implant has a preferential direction (or also several preferential directions) for cell movement and cell growth.

In this embodiment, provision is therefore made to design the lattice structure according to the invention with one or more preferential directions for the cell growth, in order to be able to control a volume growth of the implant, which can then have refractive functions, for example. The cells located in the implant can also serve to bring about a transformation of the lattice structure.

In addition to this function, an intraocular implant with lattice structure following the inventive concept can also be ring-shaped and can be applied to the anterior face of an IOL in order to keep postoperative cell growth, so-called after-cataract, away from the optical zone and to allow this safely only in a ring shape outside the optical zone. A ring-shaped intraocular implant of this kind can be used as an IOL carrier matrix and can serve to suppress after-cataract since, wherein by steering the cell growth in a defined direction, these cells are kept away from the optical zone and thus the effect of “after-cataract”, i.e. renewed clouding of the visual field, is suppressed.

Of particular interest is an embodiment of the intraocular implant according to the invention in which mechanical and/or optical properties of the implant vary spatially. They may vary depending on direction (anisotropic), but they may also vary depending on location. A combination of variations of different mechanical and/or optical properties is also expressly possible here.

For example, in one variant of this embodiment, the effective refractive index of the implant is not constant, and instead it varies depending on the location (for example the distance from a reference point and angle) within the implant. Diffraction effects can thus be specifically set, and they can be used to improve the overall optical system of the patient's eye.

The elasticity is an important feature of the implant. It is desirable, for example, if this corresponds more or less to the elasticity of the corresponding tissue type in the recipient, for example of corneal tissue, but preferably in a healthy state. For the cornea, this means that the modulus of elasticity of the material in the transplanted state should be between 1 kPa and 1000 kPa, for example between 10 kPa and 100 kPa.

Here too, it is entirely advantageous if, in a further variant of this embodiment of the intraocular implant according to the invention, the elasticity is variable depending on the position and the direction within the implant. For example, the modulus of elasticity can vary by a factor of 10 over a layer thickness of 100 μm and can thus simulate this property of the human cornea as precisely as possible. This property can likewise vary laterally, for example with a low elasticity at the center of the implant and high elasticity at the margin.

In a further embodiment of the invention, the refractive index of the material is adapted to the refractive index of the eye structure, for example of the cornea, i.e. made similar to the refractive index of corneal fluid, stromal tissue and/or the refractive index of water, such that, in the refractive embodiment of the intraocular implant according to the invention, no diffractive effects or secondary effects are to be expected, like for example “rainbow glare”, an annoying glare effect. In this respect, it is also possible, that the refractive index of the intraocular implant is matched to the refractive index of a, for example, synthetically produced liquid filling material with which the implant is filled before, during or after implantation. As an example, the refractive index of the implant material and the refractive index of a particular hyaluronic acid are matched when this liquid is used as a liquid filling material, thus minimizing disturbing optical effects.

In a further embodiment of the intraocular implant according to the invention, its material and/or lattice structure are such that the implant has an additional optical function.

Thus, in a variant of the embodiment of the intraocular implant according to the invention, a diffractive optical element or a gradient lens is present. This is achieved by a corresponding change of the optical properties, e.g. of the refractive index.

An intraocular implant is usually optically transparent in the visible spectral range. However, in a further variant of the embodiment of the intraocular implant according to the invention, part of the visible light is absorbed. This leads to a filter function for a subregion of the visible light and/or for a fraction of the visible light or for a fraction of the subregion of the visible light.

In a third variant of the embodiment of the intraocular implant according to the invention, the effective refractive power of the implant is such (i.e. the differences between the effective refractive power of the implant and of the eye structure surrounding the implant great) that it generates a refractive action on the surrounding eye structure, for example the cornea. The lattice structure of the corneal implant or generally of the intraocular implant then has an additional diffractive optical power, for example in order to provide an additional near focus.

As has already been mentioned, the implant according to the invention is therefore flexible, but it has a high level of dimensional stability in the deployed state. The implant is generally introduced in the folded state into the eye structure, and it is only there that it is deployed. It is permeable to intraocular fluids, wherein the permeability is substantially ensured by the corresponding lattice structure. It is additionally optically transparent in the visible spectral range, although in a special embodiment it can also absorb part of the visible light. It is for example produced by additive manufacturing from biocompatible materials, in particular using technically acquired collagen and/or hyaluronic acid, with an outer 3D shape precision in the μm range.

In particular embodiments of the intraocular implant, the lattice structure thereof has at least one of the following additions:
growth factors or crosslinkers;
keratocytes;
stem cells;
anti-inflammatory agents;
nanoparticles;
solvents;
membranes.

These “additions” promote certain features of the intraocular implant and favor its long-term development in the living organism. This relates to the reduction of immune reactions, the promotion of the mechanical connection between the implant and its environment, and the lasting suppression of degradation.

Growth factors, e.g. a calcium binding epidermal growth factor, or crosslinkers can thus be incorporated in the implant.

Keratocytes, which in natural form are a constituent of the stroma of the human cornea, are preferably not contained in the intraocular implant. The intraocular implant permits the diffusion of keratocytes from the surrounding tissue into the implant. However, in a particular embodiment, the intraocular implant according to the invention, in particular in the form of a corneal implant, can already contain keratocytes.

As regards stem cells, it likewise applies that these are added during the production of the intraocular implant, or else a natural colonization can take place. During production, stem cells can either be introduced into the volume and/or applied to the surface thereof.

It is known that it is possible to implant (here in the sense of transplant, since these are natural stem cells of the recipient organism or else of another donor organism) stem cells in order to heal tissue defects. In a particular embodiment of the invention, therefore, an additively manufactured, artificial therapeutic corneal implant with integrated stem cells is provided, so as to be able to treat keratoconus defects in the cornea. The stem cells can originate, for example, from the recipient himself/herself.

In one embodiment according to the invention, therefore, provision is not only made to implant an intraocular implant with an e.g. printed lattice structure, as a refractive lattice structure for example, but also to for example already colonize the latter with stem cells. In addition, such an implant can independently naturalize the printed lattice structure through the stem cells and gradually generate a more organic inner structure.

It is also known that decellularized corneal tissue (“decellularized stromal tissue”) can function as a carrier structure for stem cells (“tectonic support for stem cells”). In a further embodiment of the inventive concept of generating intraocular implants with a lattice structure, provision is made, particularly for an intraocular lens (IOL) but also for a corneal implant in the form of a lenticule to be implanted in the cornea, to use a framework produced by additive manufacture according to the invention, in order thereby to make available a carrier matrix which is populated with stem cells and which has a natural tissue growth (e.g. lens growth).

Active substances such as anti-inflammatory agents, both non-steroidal and steroidal, can also be contained in the intraocular implant as a depot with slow release of active substance, for example by being imprinted into the implant during its production process.

Moreover, in one variant, provision is made to introduce nanoparticles into the lattice structure of the intraocular implant.

Solvents can help to dissolve other additives and guarantee their transport into the implant or through the implant, to adapt the refractive index, and/or to stabilize the lattice structure.

In a further aspect of the invention, membranes can be integrated in the implant, for example in order to facilitate the colonization of the implant with cells.

In a particular embodiment of the intraocular implant according to the invention, nanoparticles are arranged at locally different concentrations in a three-dimensional (refractive) lattice structure such that there is a different refractive index in lamellar layers and/or radial zones of the implant. In this way, for example, an optical gradient lens design of the implant can be generated.

In a further particular embodiment of the intraocular implant, its dimensionally stable lattice structure is filled with a liquid (i.e. a solvent), or else the dimensionally stable lattice structure is provided to be filled with a liquid, which remains stable in the lattice structure (in particular through capillary suction) and is optically transparent.

In an example embodiment of the intraocular implant, the latter is a corneal implant which changes the outer shape of the cornea, in particular which changes the anterior and/or the posterior surface of the cornea such that a desired refractive correction is produced. A corneal implant of this type according to the invention is implantable into the cornea or between the cornea and the epithelium following the cornea, i.e. the following outer last layer of the eye, which provides protection from environmental influences.

According to the invention, provision is made to construct a corneal implant from a biocompatible and in particular transparent material such that it has a stable internal lattice structure, wherein the outer shape of the implant is configured to change the shape of the cornea, for example in order to increase the thickness of a cornea that is too thin and thus also change the anterior and/or posterior surface of the cornea and produce the refractive corrections.

It is thereby possible, for example, to correct hyperopia or myopia and/or astigmatism. It is thus possible even to generate unconventional refractive power profiles, for example bifocality, trifocality or multifocality. Such refractive power profiles can, for example, alleviate the symptoms of presbyopia and are therefore also used in intraocular lenses (IOL) (e.g. EDOF or multifocal lenses as intraocular lenses).

Moreover, in another particular embodiment of the intraocular implant, which is again a corneal implant, the latter contains pigments, which are for example introduced in such a way that an artificial iris is created.

In an advantageous configuration, the pigments can be imprinted during the process of producing the corneal implant. However, provision can be made that the pigments are introduced only after the implantation of the corneal implant, but that zones suitable for them are made ready in the corneal implant that is to be implanted. In this way, for example, an artificial pupil (iris) can be created, either for cosmetic reasons or for increasing the depth of focus by application of a small pupil diameter.

Furthermore, gradient structures, for example, can be imprinted inside a corneal implant embodied as a lenticule. Photoactive pigments (photochromatics) may be included, e.g. likewise by impression during the production process, for example in order to achieve an absorption adapted to the light intensity or to obtain a pupil size that is automatically adjustable by the light intensity.

In a further particular embodiment, the intraocular implant is an intraocular lens and/or an IOL carrier matrix. As has already been described above, such an IOL carrier matrix can be applied in a ring shape on the anterior face of an IOL or can be provided to be applied here, in order to keep cell growth (after-cataract) away from the optical zone and safely allow this only in a ring shape outside the optical zone.

In cataract operations, it is known that epithelial cells always remain in the equator of the capsular bag and may, for example, cause the symptom referred to as “after-cataract”. According to this aspect of the invention, such cells use the carrier matrix (IOL carrier matrix) produced according to the invention, which for this purpose is inserted in close contact with these epithelial cells into the capsular bag, in order to steer the cell growth into the IOL carrier matrix and in this way prevent the symptom of after-cataract.

In a way comparable to the production of the corneal implant, epithelial cells and/or stem cells or the like can be incorporated into an IOL structure according to the invention. All aspects of the corneal implant likewise apply in principle to such a structure that is to be implanted.

A method according to the invention for producing an intraocular implant, in particular for producing a corneal implant, an intraocular lens or an IOL carrier matrix, which has a dimensionally stable lattice structure, comprises at least one of the following production sequences:
(a) production of a transparent base workpiece for the intraocular implant, which base workpiece initially does not yet have the shape intended for the implant, i.e. is present in the form of a cuboid or a hemisphere, and subsequent use of a separation method, in particular cutting, for example by application of a femtosecond laser, for shape adaptation and for generating a final shape of the intraocular implant;
(b) primary forming from an initially liquid and/or gel-like mixture, in particular by molding (injection molding) or by a generative manufacturing method (3D printing, also called additive manufacture or generative manufacture);
(c) machining in order to modify the material structure, in particular for generating cavities and/or for changing chemical bonds between the constituents of the material (e.g. molecules).

Such a corneal or intraocular implant is for example produced in a single production sequence (b) by application of 3D printing.

In one embodiment of the method according to the invention for producing an intraocular implant, a transparent base workpiece for the intraocular implant is produced according to (b) and then has its shape adapted according to (a).

In a further embodiment of the method according to the invention for producing an intraocular implant, the intraocular implant produced according to (b) and/or adapted in shape according to (a) has its material structure modified according to (c).

In a further embodiment of the method according to the invention for producing an intraocular implant, a femtosecond laser keratome is used in step (a) and/or (c). The latter is suitable in particular for the machining of collagen material, in particular native collagen material (e.g. human cornea, fish scales or the like) and is therefore also a preferred example tool for adapting the shape or modifying the material structure of the intraocular implant.

In a further example embodiment of the method according to the invention for producing an intraocular implant, irradiation with light, for example from the wavelength ranges of UV radiation, of visible light or of infrared radiation, is used for the modification, in particular for an inner structuring, of the intraocular implant according to (c), this on account of the optical transparency of the material. Irradiation can lead to different types of modification of the material: to a change of the material through the effect of heat, to a change of the material through plasma formation or transformation, or to a change of the material through triggering of a chemical reaction.

This irradiation with light can be substantially homogeneous or can be dosed differently according to location, in order thereby to achieve a spatial modulation of the effect. In the case of irradiation with light for the modification, in particular for the inner structuring, of the intraocular implant, a method is for example preferred in which the light is dosed differently according to location, and its effect is thus spatially modulated, with the result that a desired structuring is obtained.

In a further embodiment of the method according to the invention for producing an intraocular implant, a chemical reaction is generated, particularly by the fact that a photosensitizer, e.g. riboflavin, which causes a chemical reaction during irradiation, which in turn leads to crosslinking of the material, is introduced beforehand into the material.

In order to generate or improve the effect, the material can contain one or more components which, as a result of irradiation, cause a desired chemical reaction.

A lithography method is for example used for the modification or inner structuring of the intraocular implant by irradiation with light. A spatially modulated increase in the crosslinking of molecules located in the material can thus be used for generating a lattice structure. The same is of course also possible in the case of scanning irradiation with focused laser light.

The structuring can also lead to the changing of optical properties of the material, for example the refractive index. It is in this way possible, for example, to generate a diffractive optical element or a gradient lens.

In a particular embodiment of the method according to the invention for producing an intraocular implant, the intraocular implant is populated artificially with cells (e.g. keratocytes) during and/or after its production.

In such a method, stem cells can be integrated at high concentration in a volume of the intraocular implant in a printing operation.

Decellularization of natural lenticules (of the cornea) is of course possible. However, subsequent recolonization with cells proves to be difficult. Therefore, in a variant of the invention, provision is made to produce lenticules artificially by additive manufacture and to integrate stem cells at a suitable concentration in the volume during the printing operation.

A variant of the method according to the invention for producing an intraocular implant is for example in which a measurement accompanying and optimizing the production is carried out during the production method. A closed-loop method is thus permitted: During the production of the intraocular implant, a measurement of a property of the implant is carried out (repeatedly) as an accompaniment to the production process, in order to use the (respective) measured value to optimize the production process, for example in order to arrive iteratively at an optimal result as regards this property of the implant.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIGS. 1A, 1B, 1C and 1D depict ocular implants according to example embodiments of the invention; and

FIG. 2 depicts a corneal implant implanted in a cornea according to an example embodiment of the invention.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

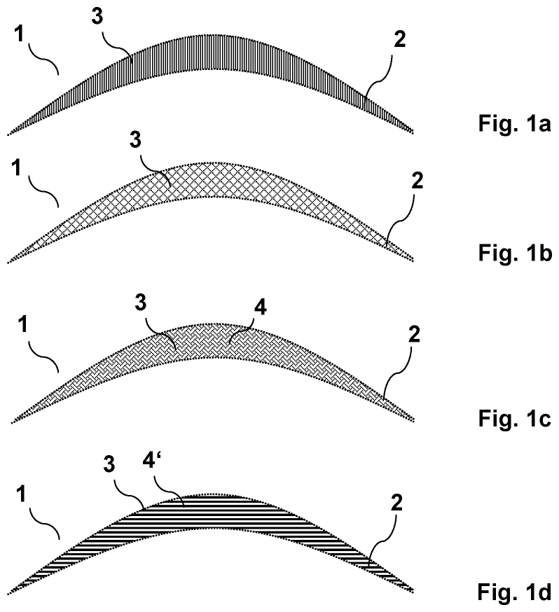

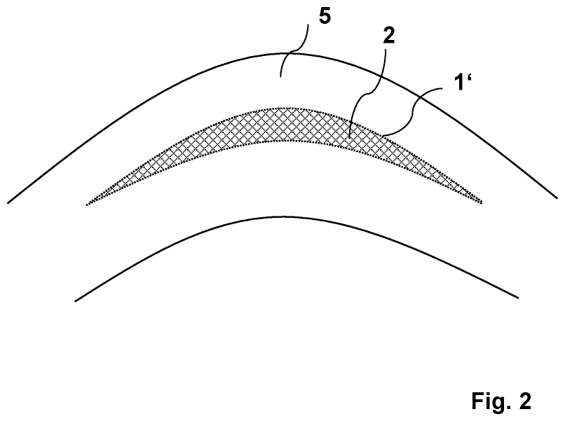

FIGS. 1A-1D and 2 show examples of corneal implants 1′ according to example embodiments of the invention, a special intraocular implant 1, in cross section, wherein, seen three-dimensionally according to the invention, always from the architecture of the lattice structure 2, there is full-surface permeability of the corneal implants 1′ for the intrastromal liquid.

FIGS. 1A-1D show various exemplary embodiments of corneal implants 1′ according to the invention in schematic cross sections through the permeable lattice structures 2. In the case of the macroscopically lamellar structure 4′ of FIG. 1D, the permeability is ensured in a perpendicular direction through a microscopically porous structure.

In particular, the outer shape of the corneal implants 1′ having the lattice structure 2 is produced here by additive manufacture, with a high three-dimensional shape precision as far as, for example, the 1μm range. On account of the elasticity properties of the biocompatible material or of the technically recovered collagen material of the printed lattice structure 2, the corneal implant 1′ remains flexible, which can be used to advantage in the implantation procedure. On the other hand, the internal lattice structure 2 ensures that the volume of the corneal implant 1′, in the deployed state after implantation, remains dimensionally stable over a long period of time.

FIG. 2 shows schematically an exemplary embodiment of a corneal implant 1′ according to the invention, with a lattice structure 2 which is designed in such a way that it permits permeability for small molecules and/or supports the mobility of endogenous cells in the implant 1′, wherein the corneal implant 1′ has been introduced into the cornea 5 of an eye.

The aforementioned features of the invention, which are explained in various exemplary embodiments, can be used not only in the combinations specified in an exemplary manner but also in other combinations or on their own, without departing from the scope of the present invention.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1.-26. (canceled)

27. An intraocular implant for implantation in an eye structure, the intraocular implant comprising:
a dimensionally stable lattice structure, the dimensionally stable lattice structure being adapted to permit permeability for small molecules, adapted to support the mobility of endogenous cells in the implant or both.

28. The intraocular implant as claimed in claim 27, wherein the lattice structure comprises a microscopically irregular framework structure.

29. The intraocular implant as claimed in claim 27, wherein the dimensionally stable lattice structure defines channels with a diameter of >1 μm and webs with a dimension of <50 μm.

30. The intraocular implant as claimed in claim 29, wherein the dimensionally stable lattice structure defines channels with a diameter of >3 μm, and webs with a dimension of <20 μm.

31. The intraocular implant as claimed in claim 27, wherein intraocular implant comprises a biocompatible material, which is arranged in the form of fibrils in lamellae in the lattice structure.

32. The intraocular implant as claimed in claim 31, wherein the biocompatible material comprises collagen.

33. The intraocular implant as claimed in claim 31, wherein the lamellae are arranged alternately at right angles to respective neighboring lamellae.

34. The intraocular implant as claimed in claim 31, wherein the lamellae are interwoven.

35. The intraocular implant as claimed in claim 27, wherein the intraocular implant is adapted for a preferential direction for cell movement or cell growth.

36. The intraocular implant as claimed in claim 27, in which mechanical properties, optical properties or both of the implant vary spatially.

37. The intraocular implant as claimed in claim 27, in which at least one of the following is true:
the material and/or lattice structure is configured such that the implant has an additional optical function;
the material and/or lattice structure is configured such that a diffractive optical element or a gradient lens is present;
the material and/or lattice structure is configured such that part of the visible light is absorbed;
the material and/or lattice structure is configured such that the effective refractive power of the implant is such that the material and/or lattice structure generates a refractive effect on a surrounding eye structure; and
the material and/or lattice structure is configured such that the effective refractive power of the implant is such that the material and/or lattice structure generates a refractive effect on the cornea.

38. The intraocular implant as claimed in claim 27, wherein the lattice structure further comprises at least one of the following additions:
growth factors;
crosslinkers;
keratocytes;
stem cells;
anti-inflammatory agents;
nanoparticles;
solvents; and
membranes.

39. The intraocular implant as claimed in claim 38, in which nanoparticles are present and arranged at locally different concentrations in the lattice structure, such that there is a different refractive index in lamellar layers, radial zones of the implant or both.

40. The intraocular implant as claimed in claim 27, wherein the dimensionally stable lattice structure is filled with a liquid, or is adapted to be filled with a liquid, wherein the liquid remains stable in the lattice structure and is optically transparent.

41. The intraocular implant as claimed in claim 40, wherein the refractive index of the liquid is matched to the refractive index of the implant material.

42. The intraocular implant as claimed in claim 27, wherein the intraocular implant comprises a corneal implant that changes the outer shape of a cornea.

43. The intraocular implant as claimed in claim 27, wherein the intraocular implant comprises a corneal implant that contains pigments, arranged such that an artificial iris is created.

44. The intraocular implant as claimed claim 27, wherein the intraocular implant comprises an intraocular lens (IOL), an intraocular lens (IOL) carrier matrix or both.

45. A method for producing an intraocular implant, for producing a corneal implant, for producing an intraocular lens or for producing an IOL carrier matrix, which has a dimensionally stable lattice structure, comprising at least one of the following production sequences:
(a) producing a transparent base workpiece for the intraocular implant, which base workpiece initially does not yet have the shape intended for the implant, and subsequently using a separation method for shape adaptation and for generating a final shape of the intraocular implant;
(b) primarily forming from an initially liquid and/or gel-like mixture, by molding, injection molding or by a generative manufacturing method or by 3D printing;
(c) machining to modify a material structure, to generate cavities and/or to change chemical bonds between the constituents of the material or molecules of the material.

46. The method as claimed in claim 45, further comprising producing a transparent base workpiece for the intraocular implant according to (b) and then adapting a shape thereof according to (a).

47. The method as claimed in claim 45, further comprising producing the intraocular implant according to (b), adapting the intraocular implant in shape according to (a), modifying the intraocular implant's material structure according to (c) or a combination of the foregoing.

48. The method as claimed in claim 45, further comprising using a femtosecond laser keratome in step (a) and/or (c).

49. The method as claimed in claim 45, further comprising using irradiation with light for the modification according to (c).

50. The method as claimed in claim 49, further comprising dosing the light differently according to location.

51. The method as claimed in claim 49, further comprising generating a chemical reaction.

52. The method as claimed in claim 51, further comprising facilitating the chemical reaction by introducing a photosensitizer beforehand into the material.

53. The method as claimed in claim 49, further comprising using lithography for the irradiation with light.

54. The method as claimed in claim 45 further comprising populating the intraocular implant artificially with cells (keratocytes) during and/or after its production.

55. The method as claimed in claim 54, further comprising integrating stem cells at a high concentration in a volume of the intraocular implant in a printing operation.

56. The method as claimed in claim 45 further comprising carrying out a measurement accompanying and optimizing the production during the production sequence.
